(19) United States
(12) Patent Application Publication
(10) Pub. No.: US 2024/0016555 A1
(43) Pub. Date: Jan. 18, 2024

(54) CONTROL OF MEDICAL FUNCTION UNITS, RELATED SYSTEMS AND METHODS

(71) Applicant: INTUITIVE SURGICAL OPERATIONS, INC., Sunnyvale, CA (US)

(72) Inventors: John W. Zabinski, Berkeley, CA (US); Boris Foelsch, Palo Alto, CA (US); Craig Gotsill, San Francisco, CA (US); Daryl Oshatz, Mountain View, CA (US)

(73) Assignee: INTUITIVE SURGICAL OPERATIONS, INC., Sunnyvale, CA (US)

(21) Appl. No.: 18/254,670

(22) PCT Filed: Nov. 29, 2021
(86) PCT No.: PCT/US21/60980

§ 371 Date: May 26, 2023

Related U.S. Application Data
(60) Provisional application No. 63/119,083, filed on Nov. 30, 2020.

(51) Int. Cl. A61B 34/00; A61M 13/00; G16H 40/63

(52) U.S. Cl. CPC A61B 34/25; A61M 13/00; G16H 40/63

(57) ABSTRACT

A system comprises a medical function unit comprising a connector interface configured to be operably coupled to control a medical function of a medical instrument, a control system operably coupled to control the medical function unit, a first user interface and a second user interface each operably coupled to the control system. The first user interface comprises one or more first control settings mapping to one or more corresponding settings of the medical function unit. The second user interface comprises one or more second control settings mapping to the one or more corresponding settings of the medical function. The second user interface is operable during a condition of the first user interface being inoperable.

### CROSS-REFERENCE TO RELATED APPLICATION

This application claims priority to U.S. Provisional Application No. 63/119,083 (filed Nov. 30, 2020) titled “CONTROL OF MEDICAL FUNCTION UNITS, RELATED SYSTEMS AND METHODS,” the entire contents of which are incorporated by reference herein.

### TECHNICAL FIELD

Aspects of the present disclosure relate to control of medical function units that provide medical function support during a medical procedure, such as for example a minimally invasive medical procedure. Further aspects of the present disclosure relate to user interfaces for such medical function unit control.

### INTRODUCTION

Minimally invasive medical procedures seek to minimize patient trauma by introducing therapeutic, diagnostic, surgical, and/or imaging instruments through small incisions or natural orifices. Such instruments can include instruments that are manually operated or instruments that are teleoperated by using a computer-assisted surgical system (sometimes referred to as a telesurgical system or robotic surgical system), in which a surgeon operates an input control unit to remotely control one or more instruments operated by a manipulator system to which the one or more instruments are coupled.

Whether operated manually or via a telesurgical system, many medical instruments are coupled to medical function units that support the instrument's clinical purpose or otherwise support the overall medical procedure. Such medical function units are sometimes referred to as auxiliary function units as they can relate to a function that supports a medical procedure, with the medical procedure (e.g., biopsy, tissue manipulation, etc.) being the main procedure. For example, electrosurgical instruments are coupled to electrosurgical energy supply units (ESUs) that provide mono- and bi-polar energy to the instrument as required. Likewise, suction/evacuation, irrigation, and/or insufflation devices that may be used during various medical procedures require operable coupling to corresponding pressurized fluid and/or vacuum sources. Similarly, endoscope instruments require supporting imaging and illumination supply units, which may be combined or in separate units. In addition, medical instruments may transmit light or other forms of electromagnetic flux (e.g., lasers), and/or provide sensing or measuring functionality, which also may rely on the support of medical function units. Thus, various medical function units are typically used during minimally invasive medical procedures to support medical instruments used at a remote site of the medical procedure in performing functions such as insufflation supply, cautery smoke evacuation, ultrasonic energy generation, imaging, illumination, irrigation, suction, and/or sensing/measuring, etc. As used herein, medical instruments include, but are not limited to, instruments used for manipulating tissue, sensing an environment (e.g., imaging, pressure, oxygen etc.), supplying fluids (e.g., irrigation fluid, pressurized gas for insufflation), evacuating fluids (e.g., smoke or irrigation fluids), and other types of instruments used to perform the medical procedure or support the medical procedure. Medical function units that support medical procedures, but that do not connect to medical instruments used for a remote medical procedure, can further include anesthesia gas supply equipment and heart-lung bypass equipment, among others.

As a result of the many different types of medical function units that can be used to support medical procedures, and the various connections (electrical cables, data cables, tubes for fluid flow, etc.) between the medical function units and the corresponding medical instruments and/or directly with a patient (e.g., anesthesia gas supply equipment and heart-lung bypass equipment), providing an integrated medical function control system and control interface to which multiple individual medical function units are connected and allowing a user to interact with the integrated control interface to coordinate the control of the different medical function units are desirable. Reference is made to International PCT Publication WO 2020/180944 A1, published Sep. 10, 2020, which is incorporated by reference herein in its entirety, which discloses various embodiments of an integrated medical function control system that employs an integrated control interface which combines various user interface control portions respectively operably coupled to multiple medical function units so as to provide a control/feedback interface that allows access to the control the settings of and receive feedback on an operational state of multiple different medical function units.

The integration of control over settings and receipt of feedback from one or more medical function units into an integrated control system and control interface of a medical system used to perform a remote medical procedure provides a robust, simplified, and overall user-friendly workflow, which can lead to greater efficiency and accuracy during medical procedures. A need exists, however, to address issues that may arise due to such integration, such as maintaining a sufficient level of independent operation of a medical function unit, the controls of which may be otherwise integrated into an overall integrated control interface of a medical function control system.

### SUMMARY

Exemplary embodiments of the present disclosure may demonstrate one or more of the above-mentioned desirable features. Other features and/or advantages may become apparent from the description that follows.

In accordance with an exemplary embodiment, the present disclosure contemplates a system comprises a medical function unit comprising a connector interface configured to be operably coupled to control a medical function of a medical instrument, a control system operably coupled to control the medical function unit, a first user interface and a second user interface each operably coupled to the control system. The first user interface comprises one or more first control settings mapping to one or more corresponding settings of the medical function unit. The second user interface comprises one or more second control settings mapping to the one or more corresponding settings of the medical function. The second user interface is operable during a condition of the first user interface being inoperable.

In another embodiment, the present disclosure contemplates a system comprising a medical function unit configured to provide a medical function during a medical procedure, the medical function being adjustable by the medical function unit. The system further comprises a first user interface operably coupled to the medical function unit, the first user interface comprising a first adjustable control setting mapped to control a parameter of the medical function, and a second user interface operably coupled to the medical function unit, the second user interface comprising a second adjustable control setting mapped to control the parameter of the medical function. The second user interface is operable during a condition of the first user interface being inoperable.

In yet another embodiment, the present disclosure contemplates a control tower of a medical system for performing a medical procedure, the control tower includes a medical function unit comprising a connector interface configured to provide a connection to a medical instrument supported by the medical function unit, and a first user interface control area comprising one or more first control settings associated with control of the medical function unit. The control tower further comprises a user interface operably coupled to the medical function unit, the user interface comprising a plurality of additional user interface control areas comprising additional control settings to adjust one or more control settings of the medical system. One of the plurality of additional user interface control areas comprises one or more additional control settings associated with control of the medical function unit, and the one or more first control settings of the first user interface control are redundant to at least some of the one or more additional control settings of the one additional user interface control area.

Yet another embodiment contemplated by the present disclosure is a method of controlling medical functions utilized in a medical procedure. The method may comprise receiving at a processor an input to power down a first control interface in an operational state of providing first control settings associated with a medical function unit of the medical system; and in response to receiving the command to power down the first control interface, outputting from the processor, a command causing control of the medical function unit to transfer to a second control interface, separate from the first control interface.

Additional objects, features, and/or advantages will be set forth in part in the description which follows, and in part will be obvious from the description, or may be learned by practice of the present disclosure and/or claims. At least some of these objects and advantages may be realized and attained by the elements and combinations particularly pointed out in the appended claims.

It is to be understood that both the foregoing general description and the following detailed description are exemplary and explanatory only and are not restrictive of the claims; rather the claims should be entitled to their full breadth of scope, including equivalents.

### BRIEF DESCRIPTION OF THE DRAWINGS

The present disclosure can be understood from the following detailed description, either alone or together with the accompanying drawings. The drawings are included to provide a further understanding of the present disclosure and are incorporated in and constitute a part of this specification. The drawings illustrate one or more exemplary embodiments of the present teachings and together with the description serve to explain certain principles and operation. In the drawings:

FIG. 1 is a functional block diagram of an embodiment of a medical function control system of the present disclosure;

FIG. 2 is a functional block diagram of another embodiment of a medical function control system of the present disclosure;

FIG. 3 is a schematic plan view of a control tower of a medical function control system in accordance with an embodiment;

FIG. 4, is a diagrammatic view of a teleoperated, computer-assisted medical system according to an embodiment;

FIG. 5 is a schematic plan view of another control tower of a medical function control system in accordance with an embodiment;

FIG. 6 is a functional block diagram of another embodiment of the present disclosure;

FIG. 7 is a functional block diagram showing various states of a primary user interface control area and a secondary user interface control area in accordance with an embodiment; and

FIG. 8A-8D show differing graphical user interface control screens in accordance with various embodiments.

### DETAILED DESCRIPTION

As used herein, medical procedures and instruments include various procedures and instruments used for manipulation of body parts (e.g., suturing, ablating, cutting, grasping, fulgurating, cauterizing, stapling, etc.), but can also include imaging, sensing, diagnostic, therapeutic instruments and procedures, as well as procedures that provide support for a minimally invasive procedure at a remote site, such as, irrigation, suction, smoke evacuation, insufflation of a body cavity, etc. Thus, for example, an endoscopic imaging instruments, irrigation instruments, insufflation instruments, evacuation suction instruments, illumination instruments, laser instruments, sensing and/or measuring instruments are considered a medical instrument within the context of the present disclosure.

Medical function units in accordance with the present disclosure include equipment used to operably connect to and control functions of a medical instrument and include, but are not limited to, insufflation units, evacuation units, electro-surgical energy generation units (sometimes referred to as ESUs), endoscopic imaging units, irrigation units, ultrasound units, and laser or other light generation units. As standalone units, conventional medical function units typically include a user interface control area that is used to provide input settings to the unit (e.g., function on/off, function power level, function type, function timing, etc.) and one or more connector interfaces that are used to operably connect the unit to one or more corresponding medical instrument it supports. A single medical function unit may supply one or more than one function. For example, a single ESU may provide monopolar energy to one or two monopolar instruments and to one or two bipolar instruments, or a single insufflation/suction unit may provide both insufflation and gas evacuation via separate gas lines. If a single medical function unit supplies and controls more than one medical function, each individual medical function unit typically has a separate user interface control portions on the medical function unit to adjust the control settings of the medical function unit. Medical function units also may include equipment that does not couple to a medical instrument used to perform a medical procedure but nonetheless provides a supportive role in the overall medical procedure. Examples of such medical function units include, but are not limited to, anesthesia supply units, heart-lung bypass support units, etc.

As discussed above, in medical systems and methods that rely on multiple medical functions to support a medical procedure, such as for example, those connecting to medical instruments that perform a medical function of a medical procedure, providing an integrated control system and control interface to control multiple ones of the medical function units being used is desirable in order to facilitate the ability to adjust control settings and to monitor the operational status/settings of the various medical function units, as well as providing an efficient and user-friendly interface for connecting medical instruments supported by the medical function units. However, integrating the control of multiple medical function units into a common control interface can pose issues, such as a loss of independent control of each medical function unit. For example, if a communication loss should occur between a medical function unit and an integrated control interface and/or if a power loss to the integrated control interface should occur, then power and/or control over the medical function unit will cease until communication is restored. This can be problematic, particularly for medical function units that may be used for a more critical medical function in support of the overall medical procedure, such as, but not limited to, for example, insufflation, anesthesia supply, or imaging. Despite how critical or not any particular medical function supporting a medical procedure may be, it may be desirable to maintain control of any such medical function units in the event an integrated control interface is unable to maintain communication with such units or other operable connection to the same.

To address these challenges, the present disclosure contemplates a medical function control system for a medical system that employs a primary, integrated control interface used to control multiple medical function units, as well as one or more secondary control interfaces dedicated to one or more medical function units and configured to maintain power and operation independently of the primary, integrated control interface should the need arise. The secondary control interfaces can have an operational state that allows the secondary control interface to take over and maintain control of the medical function unit to which it is dedicated in the event the primary integrated control interface is inoperable to do so. The secondary control interface may have user interface features (e.g., feedback, adjustable control settings, etc.) and control logic that is completely or partially redundant of the control portion of the primary, integrated control interface that is operably coupled to control the same medical function unit.

In accordance with various exemplary embodiments, the present disclosure contemplates a medical function control system in which the primary integrated control interface is part of a control system tower that provides control and a primary integrated control interface for multiple medical function units, and optionally for other operational parameters of an overall medical system, as well as co-locating the various medical function units. In this manner, a central location for the control settings and connector interfaces (e.g., ports) of multiple medical function units that are configured to provide connection to the respective instruments being supported by the medical function units can be provided as a single integrated control tower, as disclosed in WO 2020/180944 A1, incorporated by reference herein. Any secondary control interfaces that provide control of medical function units can also be provided as part of the control tower, and generally would be in a location that makes it apparent it is dedicated to its respective medical function unit.

Embodiments of medical function control systems and methods described herein may be used, for example, with computer-assisted surgical systems (sometimes referred to as robotic surgical systems) such as, but not limited to, the da Vinci Xi®, da Vinci X®, and da Vinci SP® Surgical Systems commercialized by Intuitive Surgical, Inc. But such use is not limited and the embodiments discussed herein can be used with a variety of surgical systems that utilize multiple medical function control of medical instruments, whether via a teleoperated medical instruments or a manually operated medical instruments.

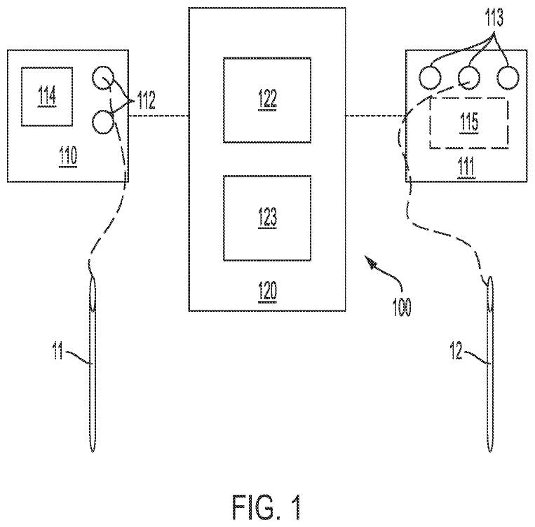

FIG. 1 illustrates a block diagram of one embodiment of a medical function control system 100 according to the present disclosure. The medical function control system 100 comprises a first medical function unit 110 and a second medical function unit 111, each configured to provide independent medical function control to one or more medical instruments 11, 12 operably connected to one or more connector interfaces 112, 113 of the respective medical function units 110, 111. The medical function units 110, 111 and the medical instruments 11, 12 can be of various types as has been discussed above. The medical function control system 100 further comprises a primary integrated control interface 120 in logical and electrical communication with both medical function units 110, 111. The primary integrated control interface 120 can employ one or more processors and control logic to control various setting parameters of the medical function units 110, 111, and includes first and second discrete user interface control areas 122, 123 each respectively assigned to provide adjustable control settings and feedback to control medical unit 110 and medical unit 111.

The first and second discrete user interface control areas 122, 123 can be part of an overall display panel and may be touchscreen or have a combination of touchscreen and mechanical control features to adjust settings of various parameters of the medical function units, which parameters change depending on the type of each medical function unit 110, 111 as those having ordinary skill in the art would understand. In addition, the first and second discrete user interface areas 122, 123 can provide feedback and information regarding settings and operating conditions of the respective medical function unit 110, 111 that each controls. The content and operation of the first and second discrete user interface control areas 122, 123 can include a variety of formats, including various graphical user interface icons, banners (which can be stationary or scrolling), etc. and the ability to present information on multiple “pages” of the interface control area by scrolling or otherwise moving through various menu functions of a graphical user interface. Those having ordinary skill in the art would appreciate a variety of forms and operational states provided by the first and second discrete user interface control areas that could be employed without departing from the scope of the present disclosure.

As further shown in the embodiment of FIG. 1, one or more of the medical function units 110, 111 of the medical function control system 100 can further include a secondary control interface 114, with a secondary user control interface area, associated with medical function unit 110 shown. The medical function unit 111 is depicted showing an optional secondary control interface having a secondary user control interface 115, as one or more of the medical function units of the overall system can include a secondary control interface depending on which medical function units it is desired to maintain control over should the primary, integrated control interface 120 be unable to provide such control. Various conditions for the loss of such ability of the primary, integrated control interface to control one or more medical function units is described further below.

The secondary user interface control areas of the secondary control interfaces 114, 115 (if any) can be completely or partially redundant of the discrete user interface control area 122, 123 to which it corresponds, i.e., based on the medical function unit each controls. Thus, in operation, a communication link, described further below, shares the settings and operational conditions between the corresponding discrete user interface control area 122, 123 of the primary, integrated control interface 120 and the relevant secondary, control interface 114, 115. In the case of a partially redundant situation, the secondary, dedicated control interface 114, 115, through a secondary user interface control area, provides at least key information and control features that may be considered more critical to the operation of particular type of medical function unit. By way of nonlimiting example, for an insufflation unit, type of surgical procedure (e.g., standard, pediatric, thoracic, bariatric, vessel harvesting, trans-anal minimally invasive, etc.), actual cavity pressure, cavity pressure setpoint, actual gas flow rate, gas flow rate setpoint, and start/stop insufflation may be among key information and control settings that the secondary user interface control area includes. For an evacuation unit, exemplary key control settings and information may include setpoint for suction pressure and actual suction pressure. For an electrosurgical energy unit, energy type and energy level settings and actual may be included as key information and control settings. For an endoscope imaging unit, type of illumination (e.g., white light, infrared, laser, etc.), image type (e.g., standard, special light spectrum, etc.), white balance, light source angle may be key information and control settings that are included. All of the above are nonlimiting and others can be included and/or some of those above may not be included.

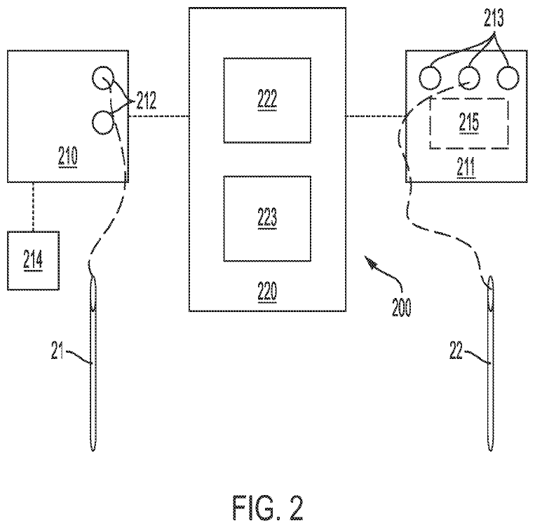

In the embodiment of FIG. 1, the secondary control interface operably coupled to a medical function unit is provided co-located or as part of the overall medical function unit, but such an arrangement is not limiting and the secondary control interface could be located in any of a variety of positions depending on a variety of factors, such as workflow considerations, access by various personnel, etc. FIG. 2 depicts an embodiment of such an arrangement, with the remaining portions and labels remaining the same as in the embodiment of FIG. 1, except as the 200 series. Those of ordinary skill in the art would appreciate that a similar arrangement can be made with any other medical function unit and its secondary, dedicated control interface. Further, it should be understood that the primary, integrated control interfaces 120, 220 can include a single user interface control area that can be operably coupled to control the settings of and provide feedback regarding an operational status of both medical function units 110, 111 and 210, 211. Such a configuration may utilize a multiple “page” screen that permits scrolling through different pages of control settings and features that correspond to each different medical function unit; also, banners, stationary or otherwise, may be used to condense space and provide information/control settings. Banners may be particularly useful to display warning and/or caution messages simultaneously with maintaining a display regarding the actual operational state of a medical function unit or while allowing change to control settings.

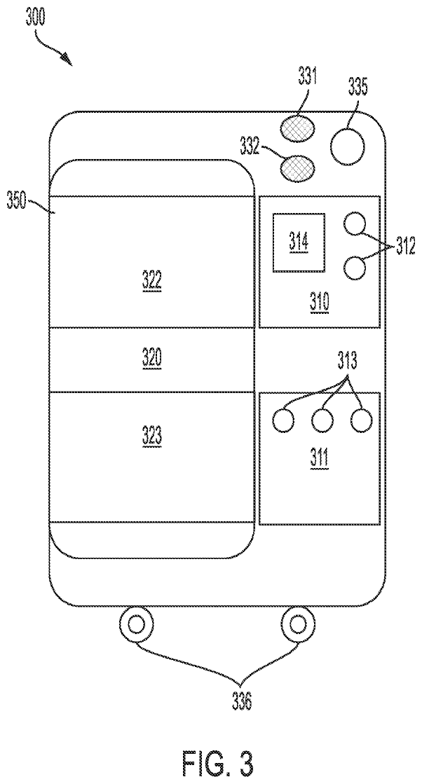

FIG. 3 illustrates yet another arrangement of a medical function control system. In the embodiment of FIG. 3, a medical function control system 300 is illustrated in which the primary, integrated control interface 320, the multiple medical function units 310, 311 (two being depicted in FIG. 3, but any number of which can be provided), and a secondary control interface 314 corresponding to and operably coupled to control medical function unit 310 provided as part of an overall control tower 350. As described above, the medical function units 310, 311 may be different types and comprise connector interfaces (e.g., ports) 312, 313 to connect with the medical instruments (not depicted) each supports. The medical function control system 300 can optionally include an additional secondary, dedicated control interface (not shown in FIG. 3) operably coupled to the medical function unit 311 or that the secondary control interface 314 is operably coupled to control medical function unit 311 as well as medical function unit 310. In the latter configuration, the secondary control interface can have differing discrete user interface control areas that are either both accessible simultaneously or that are accessible on different “pages” through a scrolling ability of what is displayed and accessed by the user at any given time. Similarly, while FIG. 3 depicts the primary, integrated control interface 320 including two discrete user interface control areas 322, 323, as described above, the areas could be combined into one with different “pages” accessible via scrolling or the like to provide access to the various control/feedback settings corresponding to each respective medical function unit 310, 311. FIG. 3 further depicts an arrangement of the different discrete user interface control areas 322, 323 being generally aligned with the medical function units 310, 311 that each discrete user interface control area 322, 323 respectively controls. Such an arrangement can facilitate use of the medical function control system 300 by providing a visual association between a discrete user interface control area and a medical function unit operably controlled by such discrete interface control area. In addition, the secondary control interface 314 is shown as co-located with the medical function unit 310 so as to clarify to a user that it controls that medical function unit. However, as discussed above, such an arrangement is nonlimiting and other arrangements can be used without departing from the scope of the present disclosure.

The control tower 350 can also include a number of other optional features to provide control of the overall system. By way of non-limiting example, a power button (on/off button) 335 is depicted in the embodiment of FIG. 3 that can be a master switch to turn on the medical function control system 300, and in turn the primary, integrated control interface 320, the medical function units 310, 311, and the secondary control interface 314. Although it is also contemplated in various embodiments to have dedicated on/off power buttons for each individual medical unit, which may be mechanical or operably as part of a touchscreen technology employed by a secondary user interface control area. Further the control tower 350 can include a speaker 331 that can provide audio feedback regarding system operation and a microphone 332 that may be wired to activate voice control of various control interfaces and/or provide external communication to an operably connected system or user located remotely from the medical function control system 300. The control tower 350 further may optionally be mobile and have wheels 336 to provide such mobility. Additionally, though not shown in FIG. 3, the control tower can include cubbies or shelves, such as on a side or back of the control tower (e.g., opposite the front where the integrated control interface is located), that are configured to receive field replaceable medical function units. Whether field replaceable or not, the control tower 350 can provide access to wiring, circuitry, and other components of the medical function units. Various other embodiments and arrangements of such a medical function control system that integrate multiple medical function units and an integrated control interface, with varied arrangements of such medical function units, their respective connector interfaces, and respective user interface control areas in an overall overlay panel, are described in International PCT Publication WO 2020/180944 A1, published Sep. 10, 2020, incorporated by reference herein.

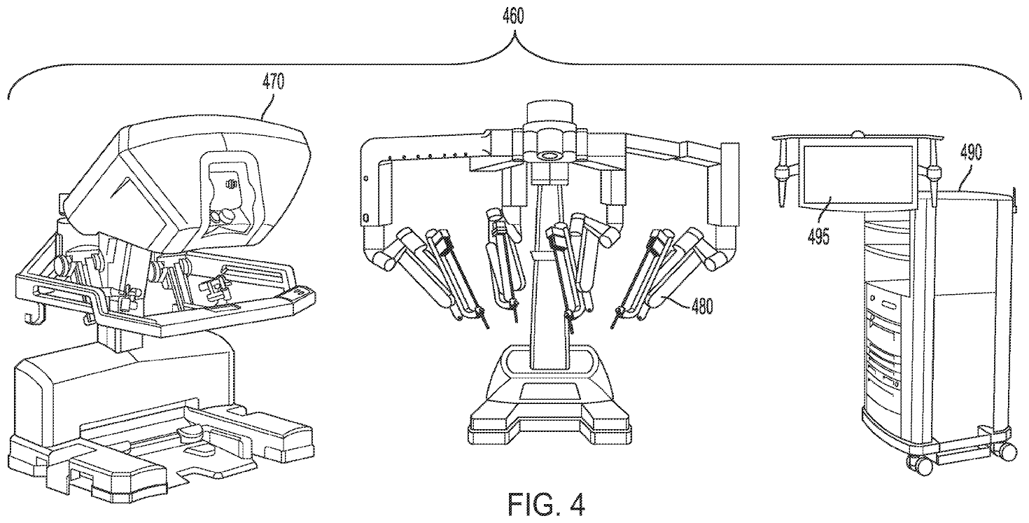

Exemplary embodiments described herein may be used, for example, with computer-assisted surgical systems (sometimes referred to as robotic surgical systems) such as, but not limited to, the da Vinci Xi®, da Vinci X®, and da Vinci SP® Surgical Systems commercialized by Intuitive Surgical, Inc. Reference is made to FIG. 4 diagrammatically illustrating the main system components of one exemplary embodiment of a computer-assisted surgical system 460 including a surgeon console 470, a manipulating system 480 and a medical function control system 490, with a display 495 as part of the medical function control system (not shown in other embodiments but can be included), which can display an image of the remote surgical site or other information concerning the medical procedure, as those having ordinary skill in the art are familiar with. In an embodiment, the medical function control system 490 can be of the type having a primary integrated control interface and the medical function units, with any corresponding secondary control interfaces, in an overall control tower, such as the control tower 350 in the embodiment of FIG. 3. But the medical function control system 490 is not so limited and can encompass any of the arrangements, distributed or otherwise, described herein, with 490 in FIG. 4 intended only to represent the general system component. The specifics of the system components 470, 480, and 490 can vary and are illustrated to represent the general components of an embodiment of a surgical system with which the medical function control systems disclosed herein may be used. As such the system components are representative, and not limiting, of other components of similar surgical systems with which the medical function control systems described herein may be used.

Those having ordinary skill in the art will appreciate that any of the embodiments described above and herein can have any number of medical function units be operably controlled by the primary, integrated control interface, and that any one or more of those medical function units can be operably coupled to a secondary control interface. Such a secondary control interface can also itself be operably coupled to more than one medical function unit and comprise differing secondary, discrete control interface areas that provide the control capabilities (e.g., adjustable settings and feedback/information) for each medical function unit, respectively.

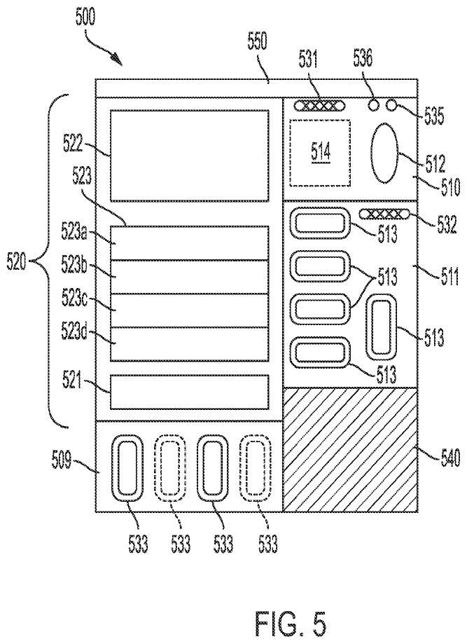

In one embodiment, it may be useful for a medical function control system to integrate as the medical function units at least an insufflation (with optional evacuation) unit and an imaging unit (e.g., configured to provide illumination and endoscopic imaging), which are generally used in a variety of surgical procedures. Additionally, providing an electrosurgical energy control unit (ESU) can also be useful as a third medical function unit. Such a combination of medical functions may have application in a variety of medical procedures, such as for example, in a variety of procedures that involve electrosurgical energy. In such case, providing a smoke evacuation unit either integrated with the insufflation unit or as a standalone unit also is desirable. FIG. 5 illustrates an embodiment of such a medical function control system 500.

As has been described with reference to other exemplary embodiments, FIG. 5 illustrates an embodiment of a medical function control system 500 in which the various medical function units and the primary integrated control interface are co-located and integrated into a control tower 550, although such an arrangement is not limiting and the various other arrangements described herein could be used as well. The view of FIG. 5 represents a schematic front view of the user interface portions of the overall medical function control system that is presented to a user to access to ports of the medical function units, settings controls corresponding to the same; components that could be included and are not shown include a display and wheels mounted to the control tower 550, similar to the tower 490 illustrated in FIG. 4. The medical function control system control tower 550 includes at least one primary integrated control interface 520 that includes multiple user interface control areas 521, 522, 523 corresponding to each of the medical function units, namely endoscopic imaging unit 509, insufflation unit 510, and electrosurgical unit 511. More specifically, user interface control area 521 is operably coupled to and configured to provide control settings for and information regarding the endoscopic imaging unit 509, which may include one or more connector interfaces to operably couple to one or more imaging and/or illumination instruments (not shown). The electrical and mechanical connections of the one or more connector interfaces are accessible through one or more connector interface ports 533 on the front face of the control tower 550. User interface control area 522 is configured to provide control settings for and information regarding the insufflation unit 510, the corresponding connector interface port 512 also being depicted and configured to receive and permit access to operably couple a connector interface with an insufflation tube device (not shown). In an embodiment, the insufflation unit may be a combined insufflation/evacuation unit and provide both insufflation and gas (e.g., smoke) evacuation functionality. User interface control area 523 is operably coupled to and configured to provide control settings for and information regarding the electrosurgical energy unit 511. Similar to the ports 512 and 533, the electrosurgical energy unit 511 includes one or more connector interface ports, with the embodiment of FIG. 5 illustrating a plurality of such ports 513, which can lead to differing types of connector interfaces to supply differing types of electrosurgical energy, such as both monopolar and bipolar to support differing types of electrosurgical instruments (not shown), with which those having ordinary skill in the art are familiar. The user interface control area 523 can further include user interface control subareas 523a-523d, each respectively corresponding to control settings for and information regarding operation of the differing electrosurgical energy connector interfaces and electrosurgical instruments connected thereto, although as explained above, instead of having differing subareas, a user interface control area can include a display that allows for scrolling through pages of user control interface subareas instead and/or include banners (scrolling or stationary) as part of the subarea to provide information/control settings.

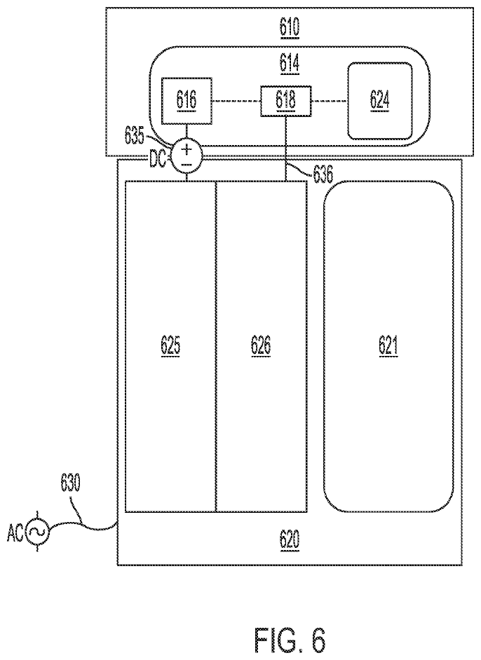

As further shown in the embodiment of FIG. 5, a secondary control interface 514 is operably coupled to the insufflation unit 510, with the secondary user interface control area being shown by the dashed lines in FIG. 5. Although not shown, one of ordinary skill in the art would appreciated that the imaging/illumination unit and/or the electrosurgical energy unit could also be operably coupled to one or more secondary control interfaces, or to the secondary control interface 514, as described above with respect to various medical function control system embodiments. Providing a secondary user interface control with at least the insufflation unit 510, however, permits the insufflation unit 510 and control thereof to remain viable in the event the operable control of the insufflation unit 510 via the primary integrated control interface 520 is not available, for example, due to a need to power down the primary integrated control interface 520 and/or due to a communication issue (e.g., such as a disruption in the communication link 636 described below in connection with the embodiment of FIG. 6 described further below) between the primary integrated user control interface 520 and the insufflation unit 510. The ability to maintain insufflation in the middle of a medical procedure is important to avoid a patient cavity collapsing while medical instruments are inserted.

Other features that may be included as part of the overall medical function control system shown in FIG. 5 include additional user interface features such as a speaker and a microphone (with speaker grill 531 and microphone grill 532 being illustrated). The locations of such features can be varied and chosen as desired. In addition, an on/off button 536 and an emergency stop button 535 can be provided and operably connected to appropriately control power to the medical function control system as a whole and corresponding medical function units. The layout and arrangement of these various additional features is exemplary and non-limiting, and those of ordinary skill in the art would appreciate various positions and sizes for such features can be modified and still be within the scope of the present disclosure. Although not depicted, the control tower 550 can also have a display mounted thereto, include a wheeled base, carry additional equipment (e.g., compressed air tanks, batteries, etc.), and/or include other storage compartments/shelving. For example, portion 540 shown in FIG. 5 can be a storage compartment, open or accessible through a shutter, door, or the like, to house the various connectors etc. for medical instruments or other devices that may be needed during a medical procedure.

In addition, the embodiment of FIG. 5 and other embodiments described herein can include other user interface control areas to display settings and/or operational status information regarding the overall system or components thereof, such as in top and/or bottom border regions, or in other areas.

As with other embodiments, the various user interface control areas and subareas of the embodiment of FIG. 5 can include graphical user interface displays and include touchscreen technology.

FIG. 6 is another functional block diagram depicting the interaction of electrical power circuitry and communications between a primary, integrated control interface and a secondary control interface of a medical function unit according to an embodiment of the present disclosure. It should be understood that the block diagram shown in FIG. 6 is not limited to any particular arrangement of the embodiments described above; for simplicity, only a single medical function unit having a corresponding secondary control interface is depicted. As discussed above, one purpose of a secondary, dedicated control interface is to provide the ability to maintain control over a medical function unit in the event of a condition occurring that may result in the power of a primary, integrated control interface being turned off and/or other signal communication disruption between the internal controller of a medical function unit and the primary integrated control interface. This can occur, for example, if a nonrecoverable fault condition of either the primary, integrated control interface or the overall medical system occurs. Under such circumstances, a user is typically prompted that such a fault condition has occurred and is provided the opportunity to power down the primary integrated control interface and restart the medical function control system. In another example, the communication link between the primary integrated control interface and the medical function unit can be disrupted, such as via a faulty cable connection or other signal interruption.

To ensure that the medical function unit operably coupled to both the primary integrated control interface and corresponding secondary control interface is able to remain operable during a power down situation, a power distribution scheme may be employed, as depicted in the embodiment of FIG. 6. As illustrated, a primary, integrated control interface 620 comprises a user interface control area 621 that can comprise a display (e.g., including touchscreen technology), which may include one or more subareas, page scrolling features, and/or banners, etc., and be able to display one or more discrete user interface areas (not separately depicted in FIG. 6 for simplicity). As described with respect to various other embodiments, the user interface control area optionally also includes mechanical control features (not shown). A power and control module 625 provides power to the primary integrated control interface 620 and various control functionality and is operably coupled to power and control various core electronic functionality through a core electronics/control module 626 of the primary, integrated control interface 620. The power supply to the primary integrated control interface can be by way of AC power connection 630, which in an embodiment may be from an AC power supply of an operating room or the like. The power and control module 625 can be a distributed system that also separately powers, for example, via a DC power supply line 635 and power module 616, the medical function unit 610 and its secondary control interface 614, including to power the secondary user interface control area 624, which can in part include a display, such as a touchscreen display. Thus, the power and control module 625 can provide a parallel circuit that takes AC power from the AC power source and provides power to the primary, integrated control interface 620 and also is converted to DC power to the power module 616. In this manner, power to the medical function unit 610, generally, and its secondary control interface 614, can be maintained even if the power module 625 powers down the primary, integrated control interface 620.

In another embodiment, in lieu of splitting the power from the AC power into the primary power module 625, a battery (not shown) could be used as a backup power to the secondary power module of the secondary control interface 614. In this case, while the primary integrated control interface 620 is powered, it can provide power to the secondary control interface 614, and optionally other components of the medical function unit 610, as well as optionally charging the battery. When the primary integrated control interface power module 625 is powered down, the power module 616 can maintain power to the secondary control interface 614 and optionally to the medical function unit generally by switching power to be supplied from the battery rather than from the AC source 630 via the power module 625 of the primary integrated control interface.

In addition, the secondary control interface 614 can include its own controller module 618 that can provide communication and control functionality between the power module 616, the medical function unit 610 components, and the user interface control area 624. The controller module 618 of the secondary control interface 614 is in communication with the core electronics/control module 626 of the primary integrated control interface 620 through a communication link 636 so that at least some of the operational parameters of the medical function unit 610 as set and monitored at the primary integrated control interface 620 can be transmitted and duplicated at the controller module 618 of the secondary control interface 614. Thus, in an embodiment, settings are synchronized between the primary control interface and the secondary control interface. For instance, during normal communications, set point changes made at the primary integrated control interface are immediately transferred to the secondary control interface, and vice versa. During loss of communications, whether due to a powering down of the primary integrated control interface or other communication loss, settings change and control is transferred to the secondary control interface, and upon reestablishing communications those new settings are then in turn pushed back to the primary integrated control interface and synchronized again.

In an exemplary embodiment, the control functions between the primary integrated control interface 620 and the medical function unit 610 and its secondary control interface 614 can utilize a CAN bus logic and architecture, although other electrical and control communications architecture may be used as well. Such CAN bus control logic and electrical signal communications architecture, as well as any others with which those having ordinary skill in the art would have familiarity, can be implemented in any of the embodiments described herein.

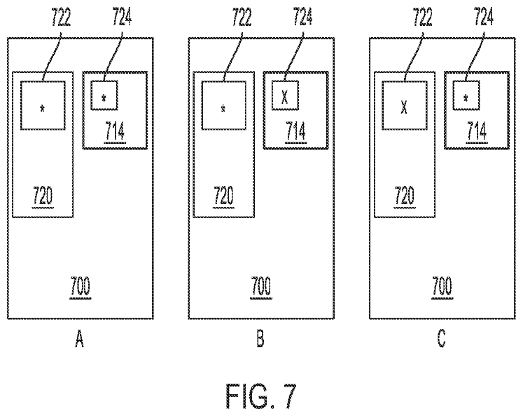

Various operational states of a medical function control system including a secondary control interface in accordance with the present disclosure will now be described. It should be understood that while the operation of only one such secondary control interface is being described for simplicity, any number of such secondary control interfaces corresponding to one or more medical function units can be used and operated in a similar manner. FIG. 7 schematically illustrates an exemplary logical workflow that may occur during a medical procedure with various states of the primary integrated control interface and a secondary control interface shown in “activated” and “deactivated” states. The “activated” state is indicated by an asterisk (*) and the “deactivated” state by an x. As used herein, “activated” refers to the relevant user interface control area enabling user interface interactions, such as showing display screen comprising information and graphics (e.g., icons) and/or enabling touchscreen functionality through the same. “Deactivate” refers to the relevant user interface control area showing a mode that indicates the user interface control area is off, such as showing a black, white, or other blank display screen. Deactivation does not necessarily mean power or other control functionality is not operable, and it should be understood that the secondary control interfaces are generally operating in the background when the primary integrated control interface and overall system is operating. Such an operational state, although showing as “deactivated” to a user, permits the second control interface to mimic the primary control interface and provide a seamless transition to its use when needed. In an embodiment, when a secondary control interface is deactivated, the control system may prevent a user from enabling interaction with the user interface control area. In other words, even if a user were to try to provide input (such as at a touchscreen display or other input feature), the user would not be able to change settings of the medical function unit. In an alternative embodiment, however, when the secondary control interface is in a deactivated state, it can become activated when a user interacts with the user interface control area (e.g., such as by initially touching the display or otherwise interacting with an input feature), even on a condition of the primary integrated control interface being operational and in a communication state with the secondary control interface. In such a situation, control settings would be transmitted to the primary integrated control interface.

Referring to FIG. 7, operational state A is a state representing initial power up of medical function control system 700, during initial power up and initialization of the system, the one or more user interface control areas 722 (one being displayed in FIG. 7) of the primary integrated control interface 720 and the secondary user interface control area 724 of the secondary control interface 714 can be activated. For example, while the various control setting and information functionality of the user interface control area 722 can be activated to be made interactive (e.g., including providing graphic user interface components on a touchscreen display), the secondary user interface control area 724 of the secondary control interface 714 can be activated by showing a splash display or otherwise changing aesthetic appearance (e.g., color, lights, etc.). Such activation of the secondary user interface control area 724 upon initial powering and initialization of the medical function control system 700 can indicate the existence of the secondary control interface 714 to a user so that it is obvious and the user is aware it exists should a power down situation or loss of communications with the primary integrated control interface 720 occur.

Operational state B in FIG. 7 shows a normal operational state of the overall medical function control system 700 in which the primary integrated control interface 720 and the secondary control interface 714 are functional and communicating. In the embodiment of FIG. 7 operational state B results in activation of user interface control area 722 (or multiple such areas with only one being shown in FIG. 7) and deactivation of secondary user interface control area 724. In operational state B, the medical function control system 700 thus is configured to encourage a user to interact with the primary integrated control interface 720 through the one or more user control interface areas 722 to alter the settings and/or receive information concerning an operational state of the medical function units operably coupled thereto. As noted above, however, in an embodiment, even while the secondary user interface control area 724 is deactivated, the overall secondary control interface 714 may be operational.

Operational state C depicts a state in which either a communication loss between the primary integrated control interface 720 and the secondary control interface 714 has occurred or the primary integrated control interface 720 has been powered down, such as in response to a non-recoverable fault of the system or another event. In operational state C, the primary user interface control area 722 is deactivated and the secondary user interface control area 724 is activated. In various embodiments, upon a user powering down the medical function control system 700, either in response to receiving a non-recoverable fault indication by the system or for another reason, the system will implement a countdown delay before powering down the primary integrated control interface 720 in order to allow activation of the user interface control area 724 and transfer of control to the secondary control interface 714. In operational state C, therefore, use of the secondary control interface 714 and its corresponding medical function unit remains so that that functionality of the medical procedure is not lost while further action is taken to recover the overall medical function control system and/or restore the communications link between the medical function unit and its secondary control interface and the primary integrated control interface.

Upon such recovery (re-powering up) of the overall medical function control system 700 and/or the restoration of the communications link, the operational states may cycle through states A and B again.

In an embodiment, the present disclosure further contemplates that the medical function control system 700 further senses various other conditions upon powering up and initialization of the overall system, including the medical function unit and its associated control interface 714 and user interface control area 724. For example, if it would be undesirable to operate the medical function unit unless a connection to a medical instrument exists, then the control system 700 can be configured to sense such condition and not put the medical function unit or its secondary control interface in an operational state for until the connection condition is met. Such a situation may also result in operational state B occurring without any initialization splash screen as indicated by operational state A occurring. In another embodiment, the primary integrated control interface could provide an error message on one or both of the user control interface area 722 or the user control interface area 724 indicating to the user that a particular condition must be satisfied before operation of the medical function unit will begin.

As described above, many permutations exist for the implementation of a medical function control system in accordance with the present disclosure, including the numbers and types of medical function units, the number and arrangements of secondary control interfaces, the arrangement and configuration of the primary integrated control interface, etc. Similarly, numerous workflow permutations to control the operational states of the primary integrated control interface, the medical function units, and any corresponding secondary control interfaces. As described above, however, the ability to maintain insufflation of a patient during a medical procedure may be of particular interest. Accordingly, a workflow for interaction for an insufflation unit, which may be integrated with an evacuation unit in an embodiment, is discussed below. Those have ordinary skill in the art would appreciate that many of the aspects of the workflow could be applied to other types of medical function units and corresponding secondary control interfaces without departing from the principles of operation discussed herein.

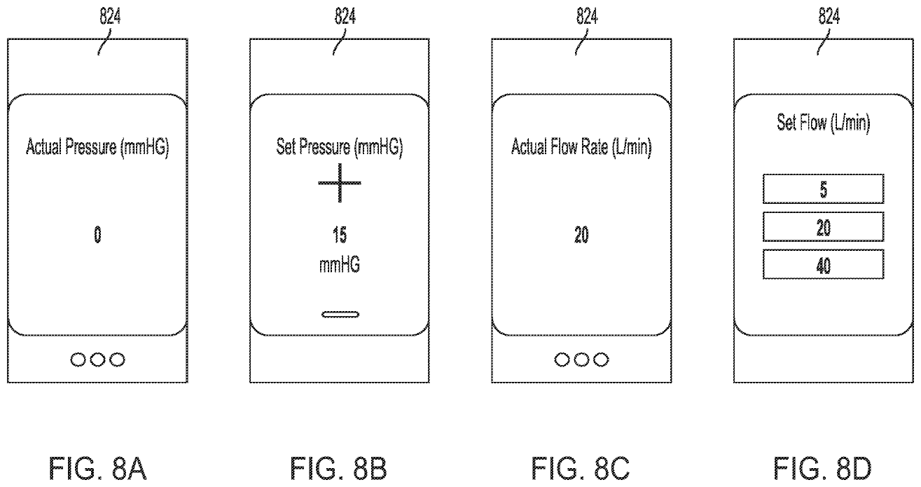

As those having ordinary skill in the art would be familiar with, an insufflation unit supplies insufflation gas (e.g., carbon dioxide) to distend a cavity so as to establish and maintain a path for entry of endoscopic medical instruments. An insufflation unit supplies the insufflation gas under a controlled and regulated pressure and flow rate, which are the general adjustable settings for the operation of an insufflation unit. When integrated with an overall medical function control system, the insufflation unit can be controlled by the primary integrated control interface, but also include, as discussed in various embodiments above, a secondary control interface, which can include a secondary user interface control area (e.g., touchscreen display) and speaker for audio feedback. The secondary control interface thus provides some redundancy, generally by providing key insufflation unit control settings interaction and information, to the primary integrated control interface. In an embodiment shown in FIGS. 8A-8D, for example, a secondary user interface control area 824 of an insufflation unit is shown in isolation and can provide pressure setting control and information, and flow rate setting control and information, with exemplary such displays being depicted. The size of the secondary user interface control area 824 may be miniaturized as compared to the corresponding user interface control area of the primary integrated control interface, and as such the different displays shown in FIGS. 8A-8D may be provided through a scrolling page functionality. The secondary user interface control area 824 can also be configured to display warning, caution, and other messages and/or information (e.g., an about page can provide serial number information, software version, service due date etc.) as would be appreciated by those having ordinary skill in the art. It can also include a page (e.g., a home page display) that has an interactive on/off feature (e.g., touchscreen icon), which can be used to turn the insufflation unit on and off independently of the primary integrated control interface. In an embodiment, the start button may be disabled from starting the insufflation unit if a valid insufflation tube device is not detected as properly installed.

Soft and hard limits of pressure and/or flow rate can be set depending on the type of mode selected for the insufflation unit (e.g., pediatric, adult, etc.). In such cases various audible or visual feedback notifications can be provided through the secondary user interface control area (or through the corresponding user interface control area of the primary integrated control interface). The control settings may further be disabled such that further increase/decrease to the pressure setting cannot occur at the user interface control area.

Moreover, if the insufflation unit includes evacuation functionality, then the secondary user interface control area can include evacuation content and pages as well. In such a configuration the insufflation tube device can include a suction evacuation tube as well. The graphical components, overall layout, contents, and functionality of the secondary user interface control area 824 that is shown in FIGS. 8A-8D is not limiting and can vary without departing from the scope of the present disclosure. In an embodiment, the size and contents of the secondary user interface control area 824 should be such that it is observable, or at least noticeable, by a user at a distance, such as across an operating room.

Outlined below are some of the general interactions and use cases of a secondary control interface for an insufflation unit. The described interactions and functionality are non-limiting and are not necessarily described in any particular order of operation. It is envisioned that other schemes can be utilized and that not all of the interactions described below may be implemented in an embodiment.

Activation/Deactivation Events

Upon power-up of the insufflation unit, if a CAN bus heartbeat message is not detected, the secondary user interface control area initializes in an activated state (i.e., the display screen will turn on and be available for user interaction). Conversely, if a CAN bus heartbeat message is detected, the secondary user interface control area initializes with a brief splash screen, as described with reference to FIG. 7, and then deactivates. In an embodiment, the secondary user interface control area can perform a self-test prior to activation when the CAN heartbeat message is not detected and can display a splash screen while performing such a self-test.

Upon loss of communications with the primary integrated control interface (i.e., through the communications link depicted in FIG. 6, if a valid insufflation tube device is properly installed at the insufflation unit, the secondary control interface is activated to show the pressure page, and the secondary control interface can be interacted with by a user to display and control flow rate and pressure settings.

If the insufflation tube device is not properly installed or is removed, an inactivity period will occur and after a predetermined time, such as about 5-15 minutes, the secondary user interface control area will be deactivated. A loss of communication event can be set on a condition of not receiving a CAN bus heartbeat message from the primary integrated control interface for a predetermined time period, which can be set and in an embodiment can range from 2-10 seconds.

If the secondary control interface of the insufflation unit receives a message from the primary integrated control interface requesting activation of the secondary user interface control area, the latter is activated. Such a request may occur, for example, at the beginning of a system power-down upon which a countdown to the termination of power of the primary integrated control interface occurs. If the secondary control interface receives a message from the primary integrated control interface requesting de-activation of the secondary user interface control area, the latter is deactivated. Such a request may occur, for example, after a mid-procedure restart has been fully completed.

If a CAN bus heartbeat message is detected and the secondary user interface control area of the insufflation unit is de-activated, touching or other sufficient contact with the secondary user interface control area activates it. The system can be set such that the initial touch does not trigger any user interface interactions (e.g., the touch will not activate the start/stop button or otherwise change any control settings). Subsequently, an inactivity period can be enforced such that if no further contact (e.g., touches or swipes) are detected for a predetermined time period (e.g., a range of 10-20 seconds or set as desired) the secondary user interface control area can be deactivated. After de-activation, and upon further re-activation, the secondary user interface control area can open to the pressure page. If a CAN bus heartbeat message is not detected and a predetermined time period (e.g., in a range of 5-15 minutes) of inactivity occurs without an insufflation tube device properly installed, the secondary user interface control area is deactivated and cannot be reactivated by touch. If on the other hand a CAN bus heartbeat message is detected and such a predetermined time period with not insufflation tube device being properly installed occur, then the primary integrated control interface can power down the insufflation unit.

If the secondary control interface detects a CAN bus heartbeat message from the primary integrated control interface but an operational mode, such as, for example, chosen from standard, pediatric, thoracic, bariatric, vessel harvesting, trans-anal minimally invasive, etc., has not been selected, the secondary user interface control area is activated to display a message or provide other feedback to select an operational mode on the primary integrated user interface control area.

Contents and Use of Pages of Secondary User Interface Control Area

When activated, the secondary user interface control area can have a home display page that it starts up in and that it returns to in the event of any inactivity (no touch/contact) of a preset time period (e.g., 5-20 seconds). In an embodiment, the home display it returns to is the actual pressure page. If insufflation is stopped, a dimming, color change, or other indication can occur to indicate that gas is not flowing. The same can occur for an evacuation page if the insufflation unit has evacuation functionality.

Other Feedback Implemented at the Secondary User Interface Control Area

Various changes in appearance of a display screen at the secondary user interface control area, lighting effects (illumination colors, blinking, flashing etc.), and sounds (e.g., through a dedicated speaker of the secondary user interface control area) can also be functional in an activated state of the secondary user interface control area. By way of non-limiting example, such indicators can be used to confirm desirable state or warn of undesirable state of a connection of an insufflation tube device or of the insufflation tube device itself (e.g., detection of leaks, contamination, and/or expiration of life of insufflation and/or evacuation tubes), a control parameter setting or actual state (e.g., pressure, flow rate, temperature), and/or a level of insufflation gas in a gas source (e.g., tank) connected to the insufflation unit. Such indicators can also be implemented when performing self-tests, to warn of non-recoverable faults, before powering the primary integrated control interface down, and/or when powering up or down the insufflation unit and its secondary control interface.

The present disclosure thus contemplates a variety of configurations, arrangements, operational states, and uses of a medical function control system that integrates control over multiple medical function units through the use of a primary integrated control interface and that also provides one or more secondary control interfaces for one or more of the medical function units to provide redundancy in control over the same should the need arise. Those of ordinary skill in the art will understand that the various medical function control systems illustrated and described with reference to the figures are nonlimiting and that other types, configurations, and/or arrangements of medical function units, with various types, numbers and/or configurations of connector interfaces and ports configured to connect to medical instrument may be envisioned without departing from the scope of the present disclosure and claims. Furthermore, based on a given surgical, therapeutic, diagnostic, etc. application, those of ordinary skill in the art will be able to determine other layouts for the medical function connector interfaces and user interface control areas.

It will also be understood by those of ordinary skill in the art that a complete computer-assisted medical system, which utilizes a medical function control system in accordance with embodiments of the present disclosure, can have various additional component parts, such as, for example, a manipulator system to which surgical instruments are configured to be mounted for use and a user control system (e.g., a surgeon console) for receiving input from a user to the control instruments mounted to the manipulator system and other medical function units and medical instruments (see FIG. 4 for example), which those having ordinary skill in the art are familiar.

Moreover, the various control interfaces and medical function units described herein should be understood to include or be configured to be controlled by computer programs, firmware, or some other form of machine-readable instructions, including an operating system, utilities, drivers, network interfaces, applications, and the like. Further, the control interfaces further include one or more computer processing elements such as a microprocessor or other circuitry to retrieve and execute software and can also include one or more memory/storage devices as would b understood by those of ordinary skill in the art. In particular it is contemplated such memory devices can be utilized to store settings and information concerning medical function units. One or more programs/software comprising algorithms to affect the various responses and signal processing in accordance with various exemplary embodiments of the present disclosure can be implemented by a processor, such as data interface module, of or in conjunction with the core processor of the primary integrated control interface and/or the secondary control interface and may be recorded on computer-readable media including computer-readable recording and/or storage media. Examples of the computer-readable recording media include a magnetic recording apparatus, an optical disk, a magneto-optical disk, and/or a semiconductor memory (for example, RAM, ROM, etc.). Examples of the magnetic recording apparatus include a hard disk device (HDD), a flexible disk (FD), and a magnetic tape (MT). Examples of the optical disk include a DVD (Digital Versatile Disc), a DVD-RAM, a CD-ROM (Compact Disc-Read Only Memory), and a CD-R (Recordable)/RW.

In various embodiments, the primary integrated control interface, the individual medical function units, and any secondary control interfaces can be arranged so as to facilitate the overall use and control of the various medical function units and to achieve a medical function control system that provides a simplified user operation and experience. Thus, in an embodiment, a medical function control system provides an integrated hub that optimizes the control settings and connector layout of the individual medical function units used during a medical procedure and provides both a primary integrated control interface for altering control settings and receiving feedback and other information pertaining to the individual medical function units and the respective medical devices to which they are connected, and least one secondary control interface.

This description and the accompanying drawings that illustrate exemplary embodiments should not be taken as limiting. Various mechanical, compositional, structural, electrical, and operational changes may be made without departing from the scope of this description and the invention as claimed, including equivalents. In some instances, well-known structures and techniques have not been shown or described in detail so as not to obscure the disclosure. Like numbers in two or more figures represent the same or similar elements. Furthermore, elements and their associated features that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment.

For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Further, this description's terminology is not intended to limit the invention. For example, spatially relative terms—such as “beneath”, “below”, “lower”, “above”, “upper”, “proximal”, “distal”, and the like—may be used to describe one element's or feature's relationship to another element or feature as illustrated in the figures. These spatially relative terms are intended to encompass different positions (i.e., locations) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. A device may be otherwise oriented (rotated 90 degrees or at other orientations) and the spatially relative descriptors used herein interpreted accordingly.

Further modifications and alternative embodiments will be apparent to those of ordinary skill in the art in view of the disclosure herein. For example, the devices and methods may include additional components or steps that were omitted from the diagrams and description for clarity of operation. Accordingly, this description is to be construed as illustrative only and is for the purpose of teaching those skilled in the art the general manner of carrying out the present teachings. It is to be understood that the various embodiments shown and described herein are to be taken as exemplary. Elements and materials, and arrangements of those elements and materials, may be substituted for those illustrated and described herein, parts and processes may be reversed, and certain features of the present teachings may be utilized independently, all as would be apparent to one skilled in the art after having the benefit of the description herein. Changes may be made in the elements described herein without departing from the spirit and scope of the present teachings and following claims.

It is to be understood that the particular examples and embodiments set forth herein are non-limiting; modifications to structure, dimensions, materials, and methodologies may be made without departing from the scope of the present teachings.

Other embodiments in accordance with the present disclosure will be apparent to those skilled in the art from consideration of the specification and practice of the invention disclosed herein. It is intended that the specification and examples be considered as exemplary only, with the following claims being entitled to their fullest breadth, including equivalents, under the applicable law.

What is claimed is:

1. A system for control of medical functions to support a medical procedure, the system comprising:
a medical function unit comprising a connector interface configured to be operably coupled to control a medical function of a medical instrument;
a control system operably coupled to control the medical function unit;
a first user interface operably coupled to the control system, the first user interface comprising one or more first control settings mapping to one or more corresponding settings of the medical function unit; and
a second user interface operably coupled to the control system, the second user interface comprising one or more second control settings mapping to the one or more corresponding settings of the medical function unit;
wherein the second user interface is operable during a condition of the first user interface being inoperable.

2. The system of claim 1, wherein the medical function unit is an insufflation unit configured to supply pressurized gas.

3. The system of claim 1, wherein the first user interface is part of a first integrated control interface configured to provide control settings mapping to the medical function unit and to another medical function unit.

4. The system of claim 2, wherein the second user interface is part of a second control interface operably independently of the first user interface.

5. The system of claim 1, wherein the first user interface is an area of an integrated user interface comprising at least one additional user interface area operably coupled to control at least one other medical function unit.

6. The system of claim 1, wherein the second user interface comprises a touchscreen display located adjacent the connector interface of the medical function unit.

7. A control tower of a medical system for performing a medical procedure, the control tower comprising:
a medical function unit comprising:
a connector interface configured to provide a connection to a medical instrument supported by the medical function unit, and
a first user interface control area comprising one or more first control settings associated with control of the medical function unit; and
a user interface operably coupled to the medical function unit, the user interface comprising a plurality of additional user interface control areas comprising additional control settings to adjust one or more control settings of the medical system,
wherein one of the plurality of additional user interface control areas comprises one or more additional control settings associated with control of the medical function unit, and
wherein the one or more first control settings of the first user interface control are redundant to at least some of the one or more additional control settings of the one additional user interface control area.

8. A method of controlling medical functions of a medical system utilized in a medical procedure, the method comprising:
receiving at a processor a command to power down a first control interface in an operational state of providing first control settings associated with a medical function unit of the medical system; and
in response to receiving the command to power down the first control interface, outputting from the processor, a command causing control of the medical function unit to transfer to a second control interface, separate from the first control interface.

9. The method of claim 8, wherein transferring the control of the medical function unit to the second control interface further comprises activating a second user interface control area to enable interaction with a user.

10. The method of claim 8, further comprising waiting a predetermined time period before powering down the first control interface in response to receiving the command to power down.

11. A system comprising:
a medical function unit configured to provide a medical function during a medical procedure, the medical function being adjustable by the medical function unit;
a first user interface operably coupled to the medical function unit, the first user interface comprising a first adjustable control setting mapped to control a parameter of the medical function; and
a second user interface operably coupled to the medical function unit, the second user interface comprising a second adjustable control setting mapped to control the parameter of the medical function;
wherein the second user interface is operable during a condition of the first user interface being inoperable.

12. The system of claim 11, wherein the condition of the first user interface being inoperable comprises a loss of power to the first user interface.

13. The system of claim 12, wherein the system is a teleoperated surgical system and the loss of power occurs in response to a manual power down of the teleoperated surgical system to clear a non-recoverable fault.

14. The system of claim 1, further comprising:
a second medical function unit configured to provide a second medical function during the medical procedure, the second medical function being adjustable by the second medical function unit,
wherein the first user interface is operably coupled to the second medical function unit; and
the first user interface comprises a second adjustable control setting mapped to control a parameter of the second medical function.

15. The system of claim 14, wherein:
the parameter of the second medical function is controllable only by the second adjustable control setting of the first user interface.

16. The system of any of claims 11-15, wherein:
the medical function unit comprises a connector interface configured transmit the medical function to a medical instrument connected to the connector interface; and
the connector interface is at a location adjacent a location of the adjustable control setting.

17. The system of claim 11-15, wherein:
the adjustable control setting is part of a touchscreen display.

18. The system of any of claims 11-15, wherein:
the medical function unit comprises the second user interface.

19. The system of any of claims 11-15, wherein:
the medical function unit is configured to provide an insufflation gas function.

20. The system of any of claims 11-15, wherein:
the first user interface and the second user interface are synchronized to a same adjustable control setting in an operable state of the first user interface and the second user interface.
